(19) United States
(12) Patent Application Publication
(10) Pub. No.: US 2026/0144459 A1
(43) Pub. Date: May 28, 2026

(54) ELECTROCARDIOGRAM BIOMETRIC DEVICE AND METHOD WITH USER ENROLLMENT

(71) Applicant: KOREA UNIVERSITY RESEARCH AND BUSINESS FOUNDATION, Seoul (KR)

(72) Inventors: Yon Dohn CHUNG, Seoul (KR); Hyubjin LEE, Seoul (KR)

(21) Appl. No.: 19/401,242

(22) Filed: Nov. 25, 2025

(30) Foreign Application Priority Data
Nov. 27, 2024 (KR) 10-2024-0171739
Oct. 23, 2025 (KR) 10-2025-0154364

(51) Int. Cl. A61B 5/117; A61B 5/318

(52) U.S. Cl. CPC A61B 5/117; A61B 5/318

(57) ABSTRACT

Disclosed is an electrocardiogram biometric authentication device and method that allows user addition. An electrocardiogram biometric authentication method performed by a computing device comprising at least a processor includes acquiring a target electrocardiogram signal of a user; generating a plurality of vectors by preprocessing the target electrocardiogram signal; generating an embedding vector by inputting the plurality of vectors to a pretrained embedding model; retrieving a similar embedding vector having a smallest distance from the embedding vector from among prestored embedding vectors; and authenticating the user based on a distance between the embedding vector and the similar embedding vector.

### CROSS-REFERENCE TO RELATED APPLICATION(S)

This application claims the benefit under 35 USC § 119(a) of Korean Patent Application No. 10-2024-0171739 filed on Nov. 27, 2024 and Korean Patent Application No. 10-2025-0154364 filed on Oct. 23, 2025 in the Korean Intellectual Property Office, the entire disclosure of which is incorporated herein by reference for all purposes.

### BACKGROUND

### 1. Field

The present invention relates to a biometric authentication system using electrocardiogram data, and more particularly, to a method of generating an electrocardiogram biometric authentication system using a deep learning model, without requiring model retraining or structure modification although a use addition (enrollment) occurs.

### 2. Description of Related Art

Today, with much personal information being digitally stored and the awareness of security being increased, there is a growing need for user authentication that involves verifying a corresponding user's identity through a specific method when the user uses a service. Traditional identity authentication methods include a universal serial bus (USB) security key or a password, however, these methods are inconvenient in that the user needs to carry a separate device or to remember a password. Also, when someone else acquires the user's security key or password, there is a risk that authentication may be possible although the person is not an authentic user. Therefore, biometric authentication that identifies and authenticates a corresponding user by analyzing physical or behavioral characteristics of the user has become widely used in recent years.

Since the biometric authentication analyzes characteristics of a corresponding user and authenticates the user, there is no need to carry a device or to memorize a password, providing high convenience and security. Today, features, such as fingerprint and iris, are commonly used for the biometric authentication. However, since these features are visible on the outside of a human body, they may be copied using photographs or silicon, leaving them vulnerable to security breaches. Therefore, recently, research is being conducted to use features within a human that are difficult to replicate as a method for the biometric authentication. Among them, an electrocardiogram, which is an electrical signal generated during cardiac activity, is easy to measure compared to other features inside the body and has unique characteristics for each individual, so it is attracting attention as a novel biometric authentication method. Also, the electrocardiogram has the advantage in terms of security since its value changes each time the electrocardiogram is measured, which differs from the conventional biometric authentication methods.

Since the first proposal of the potential for personal identification using an electrocardiogram, extensive research has been conducted to improve its accuracy. Currently, deep learning technology has been used to achieve high personal identification accuracy. However, deep learning-based techniques studied to date have the issue that there is a need to retrain a model or to modify a structure of the model to add a new user not included in training data. This indicates no consideration of user addition (enrollment), which frequently occurs in an environment in which an actual biometric authentication system is used. Therefore, there is a difficulty in directly applying the conventional deep learning-based electrocardiogram personal identification techniques to real-world electrocardiogram biometric authentication systems.

Accordingly, the present invention proposes an electrocardiogram biometric authentication system that does not require modifying or retraining of a model although a user addition occurs. The proposed system converts electrocardiogram data to an embedding vector through a deep learning model and stores the same in a vector database. Subsequently, when a user requests authentication, whether the user is registered to the system is determined by searching the database. Also, previous studies have only conducted experiments in an environment with a relatively small number of users. Therefore, the present invention demonstrates that the proposed technique works well even in an environment with a relatively large number of users.

### SUMMARY

A technical subject to be achieved by the present invention is to provide an electrocardiogram biometric authentication device and method that enables a user addition (enrollment).

According to an example embodiment, there is provided an electrocardiogram biometric authentication method performed by a computing device comprising at least a processor, the electrocardiogram biometric authentication method including acquiring a target electrocardiogram signal of a user; generating a plurality of vectors by preprocessing the target electrocardiogram signal; generating an embedding vector by inputting the plurality of vectors to a pretrained embedding model; retrieving a similar embedding vector having a smallest distance from the embedding vector from among prestored embedding vectors; and authenticating the user based on a distance between the embedding vector and the similar embedding vector.

An electrocardiogram biometric authentication device and method enabling a user addition according to an example embodiment may construct an electrocardiogram biometric authentication system suitable for a real-world environment in which a user addition frequently occurs, since there is no need to retrain a model or to modify its structure although a user addition occurs.

Also, introduction of a biometric authentication system using an electrocardiogram may improve the quality of medical and digital healthcare services through electrocardiogram data collected during biometric authentication.

### BRIEF DESCRIPTION OF THE DRAWINGS

These and/or other aspects, features, and advantages of the invention will become apparent and more readily appreciated from the following description of example embodiments, taken in conjunction with the accompanying drawings of which:

FIG. 1 illustrates an example of an electrocardiogram waveform;

FIG. 2 illustrates a data preprocessing process according to an example embodiment;

FIG. 3 illustrates the structure of a proposed model according to an example embodiment; and

FIG. 4 is a flowchart illustrating an electrocardiogram biometric authentication method according to an example embodiment.

### DETAILED DESCRIPTION

Disclosed hereinafter are exemplary embodiments of the present invention. Particular structural or functional descriptions provided for the embodiments hereafter are intended merely to describe embodiments according to the concept of the present invention. The embodiments are not limited as to a particular embodiment.

Various modifications and/or alterations may be made to the disclosure and the disclosure may include various example embodiments. Therefore, some example embodiments are illustrated as examples in the drawings and described in detailed description. However, they are merely intended for the purpose of describing the example embodiments described herein and may be implemented in various forms. Therefore, the example embodiments are not construed as limited to the disclosure and should be understood to include all changes, equivalents, and replacements within the idea and the technical scope of the disclosure.

Terms such as “first” and “second” may be used to describe various parts or elements, but the parts or elements should not be limited by the terms. The terms may be used to distinguish one element from another element. For instance, a first element may be designated as a second element, and vice versa, while not departing from the extent of rights according to the concepts of the present invention.

Unless otherwise clearly stated, when one element is described, for example, as being “connected” or “coupled” to another element, the elements should be construed as being directly or indirectly linked (i.e., there may be an intermediate element between the elements). Similar interpretation should apply to such relational terms as “between”, “neighboring,” and “adjacent to.”

Terms used herein are used to describe a particular exemplary embodiment and should not be intended to limit the present invention. Unless otherwise clearly stated, a singular term denotes and includes a plurality. Terms such as “including” and “having” also should not limit the present invention to the features, numbers, steps, operations, subparts and elements, and combinations thereof, as described; others may exist, be added or modified. Existence and addition as to one or more of features, numbers, steps, etc. should not be precluded.

Unless otherwise clearly stated, all of the terms used herein, including scientific or technical terms, have meanings which are ordinarily understood by a person skilled in the art. Terms, which are found and defined in an ordinary dictionary, should be interpreted in accordance with their usage in the art. Unless otherwise clearly defined herein, the terms are not interpreted in an ideal or overly formal manner.

Hereinafter, example embodiments will be described with reference to the accompanying drawings. However, the scope of the patent application is not limited to or restricted by such example embodiments. Like reference numerals used herein refer to like elements throughout.

The heart is the most crucial organ in the cardiovascular system, constantly contracting and relaxing to circulate blood, in order to supply nutrients and oxygen throughout the body. In this process, a tiny electrical current is generated, and a potential difference is measured by attaching two electrodes onto the skin surface, which is known as an electrocardiogram. The electrocardiogram is a tool widely used to diagnose heart disease due to its ability to diagnose the condition of the heart without requiring separate surgery. For medical purposes, a standard 12-lead method is usually employed to measure 12 types of electrocardiograms by attaching four electrodes, one to each limb, and six electrodes to the chest. In the past, measurement required complex equipment and was performed only in hospitals. However, with the development of wearable devices, such as a smartwatch and a smart ring, simple measurement has become possible.

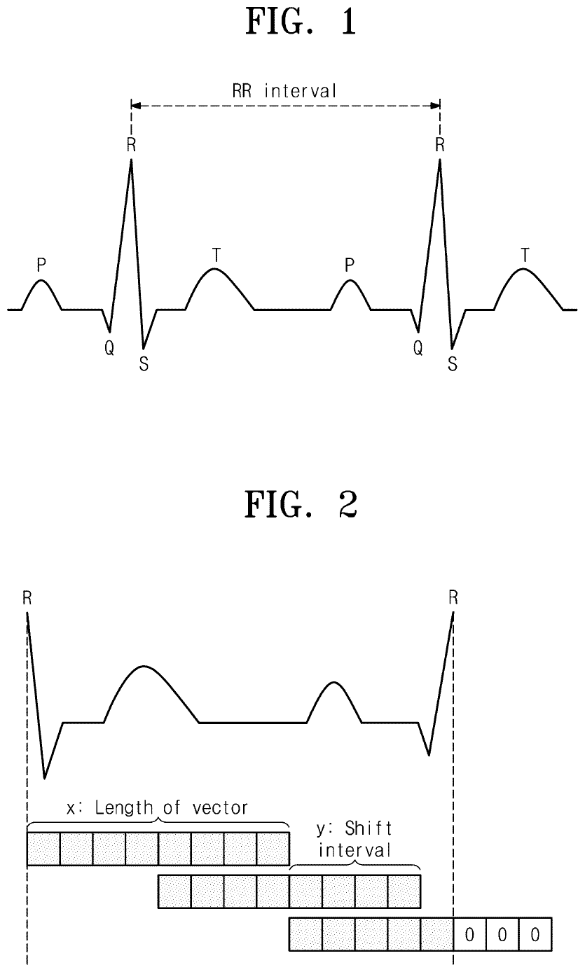

Since the heart regularly beats, a regular waveform appears on an electrocardiogram as shown in FIG. 1. The most characteristic portion of the waveform is called a PQRST-wave and a single PQRST-wave is generated per heartbeat. Here, an interval between peaks of R-waves in consecutive heartbeats is called an RR interval, and used to calculate a heart rate. The shape of this electrocardiogram waveform is unique for each user due to influence, such as a size and a location of the heart, the thickness of the heart walls, and a path of a current flowing through the heart. Therefore, research is being conducted to analyze characteristics of electrocardiogram distinct for each individual and to use the same as a method for biometric authentication.

Hereinafter, the proposed technique is described in detail.

### 1. Data Preprocessing.

An electrocardiogram is time series data in the form of a one-dimensional (1D) vector that records the potential difference over a specific period of time. For fast authentication of a biometric authentication system, an individual needs to be identified only with the shortest length electrocardiogram and a recent deep learning model has made it possible to identify an individual with only a single heartbeat-length electrocardiogram.

The present invention proposes a preprocessing method that may divide an electrocardiogram into RR intervals and may use an electrocardiogram data value as is without deformation thereof, although the length varies. Initially, as shown in FIG. 2, after cutting the electrocardiogram into RR intervals, vectors are generated each with an arbitrary length x. Here, to avoid a situation in which values are cut off and features may not be extracted, vectors are overlappingly generated while moving at intervals of y (0<y<x). If a vector exceeds an RR interval, a corresponding portion is filled with 0. In the present invention, data is preprocessed by setting x to 64 and by setting y to 32. This method maintains information on the length of the RR interval without modifying an electrocardiogram data value. However, the scope of the present invention is not limited to values of x and y, and x and y may have different values depending on example embodiments.

However, the length of the RR interval varies from user to user, and from heartbeat to heartbeat even for the same user. Therefore, the proposed data preprocessing technique generates various numbers of vectors for each electrocardiogram. This issue is addressed through the proposed model.

### 2. Proposed Model

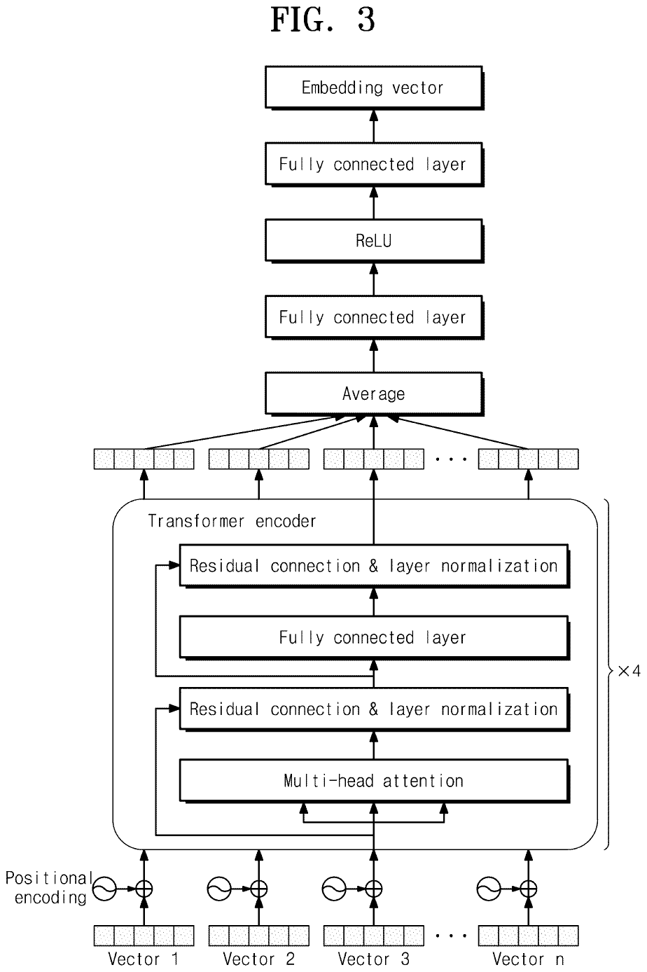

The proposed data preprocessing process converts an RR interval to a plurality of vectors with the length of 64 and uses the same as input values for a model (embedding model). That is, the length of each vector is constant, but the number of vectors is variable, which is similar to an issue commonly dealt with in the natural language processing field. Regarding an RR interval as a sentence, vectors generated in the preprocessing process may be regarded as words. Therefore, the present invention proposes a model as shown in FIG. 3 that converts an input electrocardiogram to an embedding vector using an encoder of a transformer (A. Vaswani, “Attention is all you need,” Advances in Neural Information Processing Systems, 2017), which has demonstrated excellent performance in natural language processing.

The proposed model adds location information to vectors using positional encoding in the same manner as the transformer, and uses four stacked transformer encoder blocks. A multi-head attention used in the transformer encoder uses eight heads and uses fully connected layers with 2,048 dimensions. Input vectors pass through the transformer encoder and then become new vectors with the length of 64 by maintaining the original length. These vectors are averaged into a single vector and then generated as an embedding vector with the length of 256 by passing through two fully connected layers and a single ReLU activation function. That is, the proposed model embeds the input electrocardiogram into 256 dimensions.

$$Loss = max ( 0 , {dist}_{pos} - {dist}_{neg} + margin ) \qquad [ Equation 1 ]$$

The model is trained to minimize triplet loss, which is the above equation, (F. Schroff, D. Kalenichenko, and J. Philbin, “Facenet: A unified embedding for face recognition and clustering,” in Proceedings of the IEEE conference on computer vision and pattern recognition, pp. 815-823, 2015). Based on an embedding vector of an input RR interval, distₚₒₛ denotes a Euclidean distance from an embedding vector of the same user and dist_{neg} denotes a distance from another user. Through learning, embedding vectors of the same user become closer to each other and move further apart from other users. Here, the minimum distance between other users is determined by the margin, which is set to 10 herein.

### 3. Vector Database

Unlike existing deep learning-based electrocardiogram personal identification techniques, the proposed system simply serves to convert an RR interval of an electrocardiogram to an embedding vector, without participating in a personal identification process. This embedding vector is used for storage and exploration through the vector database. Therefore, if the model is capable of well finding features that may identify an individual from an electrocardiogram and establishing a sufficient distance between individuals in the embedding dimension, user addition (enrollment) and exploration may be handled by the vector database. Therefore, although the user addition occurs, there is no need to retrain or modify the model.

Also, when many users are registered to the database, the use of an index is required for fast search. However, since electrocardiogram values are not constant for each measurement, it is difficult to use indexes such as hash or B-tree. However, the vector database may quickly find a vector most similar to a vector input with an index such as HNSW (Y. A. Malkov and D. A. Yashunin, “Efficient and robust approximate nearest neighbor search using hierarchical navigable small world graphs,” IEEE transactions on pattern analysis and machine intelligence, vol. 42, no. 4, pp. 824-836, 2018). Therefore, the proposed technique finds a vector most similar to an embedding vector of input electrocardiogram among stored vectors through HNSW, and determines the corresponding user as a registered user if the Euclidean distance between both is less than a threshold. The present invention uses Qdrant that is an opensource vector database.

Hereinafter, the experimental results are described in detail.

### 1. Experimental Environment and Dataset

Experiments were conducted on a personal computer (PC) equipped with Intel i7-9700KF 3.60 GHz CPU, 32 GB RAM, and NVIDIA Geforce RTX 3080Ti GPU. The. proposed technique was implemented using a deep learning framework, Pytorch 2.3.1, in Python 3.12.4 and CUDA 12.6.

For training of the model, an initial learning rate of Adam optimization was set to 0.001, and the learning rate was multiplied by 0.999 for each single epoch, for maximum of 5,000 epochs. The batch size was 32 and a dropout rate for preventing overfitting was set to 0.1.

PhysioNet/Computing in Cardiology Challenge 2017 (PhysioNet2017), which was provided by the competition of classifying atrial fibrillation by analyzing electrocardiograms, was used as the dataset for the experiment. In PhysioNet2017, electrocardiogram data of 8,528 users measured at various lengths from 9 seconds to 60 seconds, was labeled into four classes, normal, atrial fibrillation, other abnormality, and noise. In the present invention, among data of 5,076 users with normal signals, electrocardiogram data of 2,500 users with long lengths was divided into RR intervals and used for model training and experiment.

### 2. Experimental Results

To verify whether the proposed technique may identify individuals with high accuracy even in an environment with a large number of users, five RR intervals for each of 2,000 users were left as experimental data for accuracy evaluation and the rest were used for training. Referring to Table 1 showing the results of comparing personal identification accuracy through electrocardiograms, Yi et al. (P. Yi, Y. Si, W. Fan, and Y. Zhang, “Ecg biometrics based on attention enhanced domain adaptive feature fusion network,” IEEE Access, 2023) showed the significant decrease in accuracy when the number of users increased even slightly, and Yuniarti et al. (A. R. Yuniarti, S. Rizal, and K. M. Lim, “Single heartbeat ecg authentication: a 1d-cnn framework for robust and efficient human identification,” Frontiers in Bioengineering and Biotechnology, vol. 12, p. 1398888, 2024) showed 100% accuracy for 18 users and 99.7% identification accuracy for 218 users. In contrast, the proposed technique achieved high personal identification accuracy of 99.87% in electrocardiogram data from 2,000 users, nearly ten times. Therefore, the proposed technique demonstrates its effectiveness in an environment with a large number of users.

TABLE 1

| Technique | Dataset | Number of users | Accuracy |
|---|---|---|---|
| Yi et al. | ECG-ID | 90 | 94.26% |
| (2023) | Heartprint | 199 | 54.33% |
| Yuniarti et al. | NSRDB | 18 | 100% |
| (2024) | MIXED-3 | 218 | 99.7% |
| Proposed technique | PhysioNet2017 | 2,000 | 99.87% |

Also, the conventional techniques train the model to assign a class to each individual and to classify the same. Therefore, adding a user requires adding a class, which leads to modifying the structure of the model or retraining the model. On the other hand, the proposed technique allows a user addition without retraining the model, so the corresponding experiments were conducted as shown in Table 2. Identification accuracy for all stored users was measured with adding 500 users to the vector database, excluding 2,000 users previously used for training in electrocardiogram data of 2,500 users used in the present invention. Referring to Table 2 showing identification accuracy of the proposed technique according to the user addition, the proposed technique maintained high accuracy for the user addition even when the model was not retrained.

TABLE 2

| Number of added users | Total number of users | Accuracy |
|---|---|---|
| 0 | 2,000 | 99.87% |
| 250 | 2,250 | 99.82% |
| 500 | 2,500 | 99.36% |

The present invention proposes an electrocardiogram-based biometric authentication system that allows user addition using the vector database. Unlike the conventional techniques, a personal identification and authentication process is performed by not the model but the vector database, so there is no need to retrain the model or to modify the structure of the model although the user addition occurs. Also, the experiments have demonstrated that the proposed technique maintains high accuracy even when there are many users, while allowing the user addition.

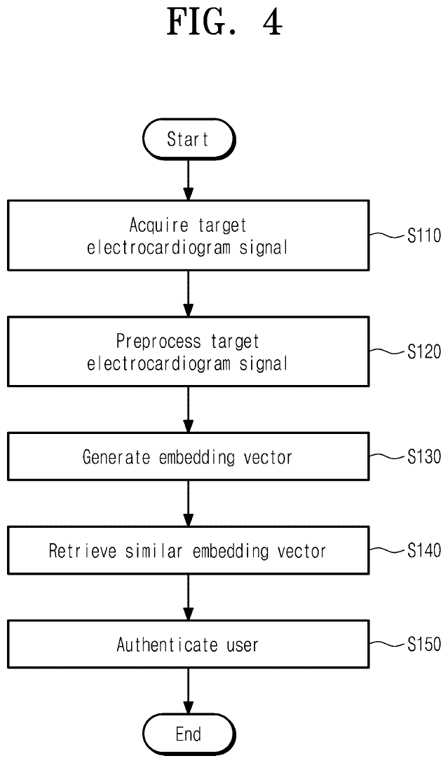

FIG. 4 is a flowchart illustrating a biometric authentication method according to an example embodiment.

Referring to FIG. 4, the biometric authentication method may be performed by a computing device including at least a processor and/or memory. That is, at least some of operations constituting the biometric authentication method may be understood as operations of a processor included in the computing device. In this case, the computing device may be referred to as a biometric authentication device. The computing device may include, for example, a personal computer (PC), a server, a tablet PC, and a laptop computer, and depending on example embodiments, may also refer to a device separately provided for biometric authentication. Also, the computing device may be implemented as a single device and/or may be implemented as a plurality of devices to construct a distributed environment. In the following description of the biometric authentication method, detailed description that overlaps the previous description will be omitted.

In operation S110, a target electrocardiogram signal of a user is acquired. The target electrocardiogram signal may have a predetermined size (length), and may be received from a measurement device that measures an electrocardiogram of the user. Depending on example embodiments, the measurement device and a computing device that performs biometric authentication may be implemented as a single device.

In operation S120, preprocessing is performed on the target electrocardiogram signal. For preprocessing, a portion is extracted from the target electrocardiogram signal. Here, the extracted portion may correspond to an RR interval, and the extracted signal may be referred to as an RR interval signal. Then, a plurality of vectors may be generated by dividing the RR interval signal. Here, the plurality of vectors all have the same length, and division is performed by applying a shift interval (which may also be referred as sliding window) less than the vector length such that adjacent vectors overlap by a predetermined interval. Also, if the length of a last vector is less than the length of a predetermined vector, the remaining portion may be filled with 0 (i.e., zero-padding). As a result of the above-described preprocessing process, the plurality of vectors with the predetermined length are generated.

In operation S130, an embedding vector for the target electrocardiogram signal is generated. The embedding vector may be generated by inputting the plurality of vectors to a pretrained embedding model. The embedding model refers to a transformer encoder-based model, and may represent a model pretrained using a predefined loss function such that embedding vectors for electrocardiogram signals of the same user are located adjacent to each other in the embedding space (i.e., have close distance), and embedding vectors for electrocardiogram signals of different users are located far apart in the embedding space (i.e., have large distance).

Specifically describing a process of generating an embedding vector, if positional encoding is applied to each of a plurality of vectors to add location information and then input to a transformer encoder, a plurality of output vectors each having the same length as that of each of the plurality of vectors are generated. The plurality of output vectors pass through an average layer, at least one fully connected layer, and an activation function, and a single embedding vector is derived.

An exemplary transformer encoder includes four blocks. The four blocks may include a multi-head attention layer, a first residual connection and layer normalization layer, a fully connected layer, and a second residual connection and layer normalization layer. However, the scope of the present invention is not limited to the structure of the transformer encoder proposed herein. Depending on example embodiments, the structure or parameters may vary.

In operation S140, a similar embedding vector is retrieved. The similar embedding vector having a smallest distance (e.g., Euclidean distance) from the generated embedding vector is retrieved from the vector database (or from among previously stored embedding vectors). Embedding vectors of previously registered users may be stored in the vector database.

In operation S150, whether to authenticate the user is determined. Whether to authenticate the user may be determined based on a distance between the embedding vector of the user and the similar embedding vector. For example, if the distance between the two embedding vectors is less than (or less than or equal to) a predetermined threshold, the user may be determined as a pre-registered user. If the distance between the two embedding vectors is greater than (or greater than or equal to) the predetermined threshold, the user may be determined as not the pre-registered user.

The device described above can be implemented as hardware elements, software elements, and/or a combination of hardware elements and software elements. For example, the device and elements described with reference to the embodiments above can be implemented by using one or more general-purpose computer or designated computer, examples of which include a processor, a controller, an ALU (arithmetic logic unit), a digital signal processor, a microcomputer, an FPGA (field programmable gate array), a PLU (programmable logic unit), a microprocessor, and any other device capable of executing and responding to instructions. A processing device can be used to execute an operating system (OS) and one or more software applications that operate on the said operating system. Also, the processing device can access, store, manipulate, process, and generate data in response to the execution of software. Although there are instances in which the description refers to a single processing device for the sake of easier understanding, it should be obvious to the person having ordinary skill in the relevant field of art that the processing device can include a multiple number of processing elements and/or multiple types of processing elements. In certain examples, a processing device can include a multiple number of processors or a single processor and a controller. Other processing configurations are also possible, such as parallel processors and the like.

The software can include a computer program, code, instructions, or a combination of one or more of the above and can configure a processing device or instruct a processing device in an independent or collective manner. The software and/or data can be tangibly embodied permanently or temporarily as a certain type of machine, component, physical equipment, virtual equipment, computer storage medium or device, or a transmitted signal wave, to be interpreted by a processing device or to provide instructions or data to a processing device. The software can be distributed over a computer system that is connected via a network, to be stored or executed in a distributed manner. The software and data can be stored in one or more computer-readable recorded medium.

A method according to an embodiment of the invention can be implemented in the form of program instructions that may be performed using various computer means and can be recorded in a computer-readable medium. Such a computer-readable medium can include program instructions, data files, data structures, etc., alone or in combination. The program instructions recorded on the medium can be designed and configured specifically for the present invention or can be a type of medium known to and used by the skilled person in the field of computer software. Examples of a computer-readable medium may include magnetic media such as hard disks, floppy disks, magnetic tapes, etc., optical media such as CD-ROM's, DVD's, etc., magneto-optical media such as floptical disks, etc., and hardware devices such as ROM, RAM, flash memory, etc., specially designed to store and execute program instructions. Examples of the program instructions may include not only machine language codes produced by a compiler but also high-level language codes that can be executed by a computer through the use of an interpreter, etc. The hardware mentioned above can be made to operate as one or more software modules that perform the actions of the embodiments of the invention and vice versa.

Although the present invention is described with reference to the example embodiments illustrated in the drawings, it is provided as an example only and it will be apparent to one of ordinary skill in the art that various alterations and modifications in form and details may be made in these example embodiments without departing from the spirit and scope of the claims and their equivalents. For example, suitable results may be achieved if the described techniques are performed in a different order, and/or if components in a described system, architecture, device, or circuit are combined in a different manner, and/or replaced or supplemented by other components or their equivalents. Therefore, other implementations, other example embodiments, and equivalents are within the scope of the following claims.

What is claimed is:

1. An electrocardiogram biometric authentication method performed by a computing device comprising at least a processor, the electrocardiogram biometric authentication method comprising:
acquiring a target electrocardiogram signal of a user;
generating a plurality of vectors by preprocessing the target electrocardiogram signal;
generating an embedding vector by inputting the plurality of vectors to a pretrained embedding model;
retrieving a similar embedding vector having a smallest distance from the embedding vector from among prestored embedding vectors; and
authenticating the user based on a distance between the embedding vector and the similar embedding vector.

2. The electrocardiogram biometric authentication method of claim 1, wherein the generating of the plurality of vectors comprises:
extracting an RR interval of the target electrocardiogram signal; and
generating the plurality of vectors having the same length by dividing the extracted RR interval.

3. The electrocardiogram biometric authentication method of claim 2, wherein the generating of the plurality of vectors having the same length comprises dividing the extracted RR interval such that adjacent vectors overlap by a predetermined interval.

4. The electrocardiogram biometric authentication method of claim 3, wherein the generating of the plurality of vectors having the same length comprises performing zero-padding such that the length of a last vector among the plurality of vectors has a predetermined length.

5. The electrocardiogram biometric authentication method of claim 4, wherein the generating of the embedding vector comprises:
adding location information by applying positional encoding to each of the plurality of vectors;
generating a plurality of output vectors by inputting the plurality of vectors with the added location information to a transformer encoder; and
generating the embedding vector by allowing the output vectors to pass through an average layer, a first fully connected layer, an activation function, and a second fully connected layer.

6. The electrocardiogram biometric authentication method of claim 5, wherein the similar embedding vector has the smallest Euclidean distance from the embedding vector.

7. The electrocardiogram biometric authentication method of claim 6, wherein the authenticating of the user comprises determining the user as a registered user if the distance between the similar embedding vector and the embedding vector is less than a predefined threshold.
